(19) United States
(12) Patent Application Publication
(10) Pub. No.: US 2022/0192187 A1
(43) Pub. Date: Jun. 23, 2022

(54) COMPOSITE VIRUCIDAL FILTER MEDIA

(71) Applicant: UOP LLC, Des Plaines, IL (US)

(72) Inventors: Chunqing Liu, Arlington Heights, IL (US); Chaoyi Ba, Schaumburg, IL (US); Xueliang Dong, Schaumburg, IL (US); Robert L. Bedard, McHenry, IL (US)

(21) Appl. No.: 17/449,471

(22) Filed: Sep. 30, 2021

Related U.S. Application Data
(60) Provisional application No. 63/129,981, filed on Dec. 23, 2020.

(51) Int. Cl. A01N 25/10; A01N 59/20; A01N 59/16; B01J 20/26; B01J 20/24; B01J 20/02; B01J 20/28; B01J 20/32; A61L 9/014; B01D 39/16

(52) U.S. Cl. CPC A01N 25/10; B01D 2239/0492; A01N 59/16; B01J 20/261; B01J 20/24; B01J 20/0237; B01J 20/0244; B01J 20/28023; B01J 20/3293; B01J 20/321; B01J 20/3289; A61L 9/014; B01D 39/1623; A61L 2209/22; A61L 2209/14; B01D 2239/0407; B01D 2239/0442; B01D 2239/10; A01N 59/20

(57) ABSTRACT

A composite virucidal filter media is described. The filter media comprises a fibrous substrate comprising a plurality of intermingled fibers, a low cost, nontoxic, hydrophilic polymer without acidic functional groups deposited on a surface of the fibers without the formation of a continuous coating layer on the substrate, and a virucidal metal, a virucidal metal-containing compound, or combinations thereof deposited on the surface of the fibers comprising the hydrophilic polymer without acidic functional groups. The hydrophilic polymer without acidic functional groups can be charged or non-charged. Methods of making virucidal fibrous filter media are also described.

### RELATED APPLICATIONS

This application claims the benefit of U.S. Provisional Patent Application Ser. No. 63/129,981 filed Dec. 23, 2020, the entirety of which is incorporated herein by reference.

### BACKGROUND OF THE INVENTION

Coronavirus disease 2019, known as “Covid-19”, is caused by severe acute respiratory syndrome coronavirus 2 (SARS-CoV-2). It has created into a global pandemic resulting in enormous numbers of illnesses requiring hospitalization and large numbers of deaths. Accordingly, the pandemic has highlighted the need for physical or chemical agents that are capable of deactivating or destroying viruses like the SARS-CoV-2 virus.

Transmission of the virus is primarily respiratory in nature. The SARS-CoV-2 virus is about 100-200 nm in diameter. The club-shaped glycoprotein spikes on the surface of the spheres give it a crownlike appearance. The glycolated S protein spikes latch onto human calls and undergo a structural change that allows the viral membrane to fuse with the cell membrane.

High efficiency particulate air (HEPA) filters, heating and air conditioning (HVAC) filters, masks, and other strategies have been used in attempts to control the spread of the virus.

Virucidal treatments often rely on metallic nanoparticles of silver, copper, zinc dispersed on the surface of fibrous media. It was reported that chitosan/Ag nanoparticles could kill virus and bacteria (A Review on Antimicrobial Chitosan-Silver Nanocomposites: A Roadmap Toward Pathogen Targeted Synthesis, International Journal of Polymeric Materials and Polymeric Biomaterials, 64:9, 448-458, 2015). It was also reported that ZnO/glass fiber filter could kill virus in water (Li X. (2010), Applications for nanomaterials in critical technologies, PhD thesis).

US 2018/0368401 disclosed textiles having antimicrobial properties. U.S. Pat. No. 6,872,241 disclosed anti-pathogenic air filtration media and air handling devices having protective capabilities against infectious airborne microorganisms. Both disclosed the use of cross-linked charged polymers on fibers.

UV treatment has also been used to kill the virus.

However, there remains a need for filters with virucidal properties, particulate rejection property, and high air permeance for HEPA, HVAC, and other applications.

### DESCRIPTION OF THE INVENTION

SARS-CoV-2 is a large, enveloped virus, the lipid envelope of which is particularly sensitive to biocides. For purposes of this application, “virucidal” means a material that is one that destroys or inactivates “the virus responsible for COVID-19” or “the COVID-19 virus”.

A composite virucidal filter media has been developed. The filter media comprises organic or inorganic fibers having a plurality of intermingled fibers, a low cost, nontoxic, hydrophilic polymer without acidic functional groups, and a virucidal metal, a virucidal metal-containing compound, or combinations thereof deposited thereon. The organic fibers can be made from polyethylene, polypropylene, or cellulose, for example. The inorganic fibers can be made from glass or carbon, for example. The virucidal metal can be Ag, Zn, Cu, for example. The virucidal metal-containing compounds can be zinc oxide (ZnO), zinc hydroxide (Zn(OH)₂), zinc hydroxy carbonate, zinc hydroxy sulfate, copper oxide (CuO), copper hydroxide (Cu(OH)₂), copper hydroxy carbonate, copper hydroxy sulfate, or a combination thereof, for example. The hydrophilic polymer without acidic functional groups can be charged or non-charged polymer and can be chitosan, sodium alginate, potassium alginate, sodium hyaluronate, potassium hyaluronate, or polyvinyl alcohol, for example.

Face masks and air filters made from nano-diameter fibers of inert polymers such as polypropylene (PP) are widely used. PP fibers have a number of important benefits. They are very effective in filtering fine particulate due to a high electrostatic charge, while maintaining very low resistance and a high loading capacity. They also have high surface area which increases the level of the charge. They absorb little or no moisture and do not hydrolyze in water. They have good wear resistance and resistance to sunlight, and they are not attacked by mildew, mold, or bacteria. In addition, they are light weight and low cost.

However, the surface of PP fibers does not have any functional groups, rendering it inert. Consequently, it is difficult to coat antivirus metal and metal-containing particles on the surface of PP fibers.

Previous work demonstrated the ability to obtain good adhesion and coating stability between chitosan and inert polymers such as polyethylene (PE), PP, and blends of PE and PP. See U.S. Ser. No. 17/389,032, filed on Jul. 29, 2021, entitled Ionically Conductive Thin Film Composite Membranes for Energy Storage Applications, which is incorporated herein in its entirety.

Suitable polymers for the fibers include, but are not limited to, polyethylene (PE), polypropylene (PP), polyamide (such as Nylon 6, Nylon 6,6, and Aramid), polyacrylonitrile, polyethersulfone, polysulfone, poly(ether ketone), polyimide, polyvinylidene fluoride, carbon, polycarbonate, polyester, cellulose acetate, cellulose triacetate, polybenzimidazole, cellulose, acrylic, glass, or combinations thereof.

A hydrophilic polymer without acidic functional groups is deposited on the surface of the fibers so that it does not form a continuous coating layer. Because the hydrophilic polymer without acidic functional groups does not form a continuous coating, portions of the fiber surface exposed to the electrostatic charge of the fiber can attract particulate. Suitable hydrophilic polymers without acidic functional groups include, but are not limited to, a polysaccharide polymer, a polyvinyl alcohol, a poly(ethylene glycol), a poly(vinyl pyrrolidone), a polyacrylamide, a polyethyleneimine, or combinations thereof. Suitable polysaccharide polymers include, but are not limited to, chitosan, sodium carrageenan, potassium carrageenan, sodium alginate, potassium alginate, sodium hyaluronate, potassium hyaluronate, dextran, pullulan, carboxymethyl curdlan, chitin, or combinations thereof. The hydrophilic polymer without acidic functional groups may interact with the virucidal metal, virucidal metal-containing compound, or combinations thereof to form a water insoluble material.

The virucidal metal, virucidal metal-containing compound, or combinations thereof are deposited on the surface of the fibers where the hydrophilic polymer is located. Without the presence of the hydrophilic polymer on the fibers, the metal and/or metal-containing compounds cannot be deposited on the fibers or can be washed off easily. By “metal-containing compounds,” we mean a compound that contains one or more metal elements bonded to one or more nonmetallic elements. The metal atom in the metal-containing compound acts as the cation in the compound and is bonded to a nonmetallic anion or an ionic group. Suitable metal-containing compounds include, but are not limited to, metal oxides, metal hydroxides, hydrous metal oxides, metal sulfates, hydrated metal sulfates, metal halides, hydrated metal halides, metal nitrates, hydrated metal nitrates, hydroxy double salts (e.g., metal hydroxy carbonate or metal hydroxy sulfate), or combinations thereof. Suitable metals (elemental or as part of the metal-containing compounds) include, but are not limited to, Ag, Cu, Zn, Ni, Ti, Sn, Bi, W, Fe, Al, Ru, Ir, Mn, Ca, or combinations thereof.

In one embodiment, the hydrophilic polymer without acidic functional groups comprises chitosan, sodium carrageenan, potassium carrageenan, sodium alginate, potassium alginate, sodium hyaluronate, potassium hyaluronate, or combinations thereof, and the metal or the metal in the metal-containing compound comprises Ag, Cu, Zn, or combinations thereof.

One aspect of the invention is a method of making a virucidal fibrous filter media. In one embodiment, the hydrophilic polymer without acidic functional groups is dissolved in water or an acidic aqueous solution to form an aqueous polymer solution. One example of a suitable acidic aqueous solution is an acetic acid aqueous solution containing about 0.1 to about 4 wt % of acetic acid in water. In one embodiment, the aqueous polymer solution comprises a polymer with a concentration in a range of about 0.005 to about 5 wt %, or about 0.01 to about 1 wt %, or about 0.05 to about 0.5 wt %.

The aqueous polymer solution containing the hydrophilic polymer without acidic functional groups is applied onto a fibrous filter substrate comprising a plurality of intermingled fibers or onto loose fibers to deposit the hydrophilic polymer without acidic functional groups on the surface of the intermingled fibers or the loose fibers so that the hydrophilic polymer without acidic functional groups does not form a continuous coating layer on the substrate. The aqueous polymer solution containing the hydrophilic polymer without acidic functional groups is applied onto a fibrous filter substrate comprising a plurality of intermingled fibers or onto loose fibers by processes including but not limited to, dip coating, impregnating, brushing, spraying, meniscus coating, roll coating, spin coating, flow coating, filtration, or combinations thereof. One method to achieve the deposition of the hydrophilic polymer without acidic functional groups on the surface of the intermingled fibers or the loose fibers without the formation of a continuous coating layer on the substrate is to control the polymer concentration of the aqueous polymer solution. A polymer concentration of about 0.005 to about 0.5 wt % will provide a hydrophilic polymer without acidic functional groups deposited intermingled fibers or the loose fibers without the formation of a continuous coating layer on the substrate. Another method to achieve the deposition of the hydrophilic polymer without acidic functional groups on the surface of the intermingled fibers or the loose fibers without the formation of a continuous coating layer on the substrate is the use of a phase inversion technique, which is to contact the wet aqueous polymer solution deposited on the intermingled fibers or the loose fibers with a non-solvent for the polymer to form a non-continuous porous polymer coated intermingled fibers or the loose fibers. The aqueous polymer solution for the phase inversion technique has a polymer concentration in a range of about 0.1 to about 5 wt %. The non-solvent for the polymer is used to precipitate out the polymer from the wet aqueous polymer solution deposited on the intermingled fibers or the loose fibers without the formation of a continuous coating layer. The non-solvent for the polymer can be selected from any solvent that has good miscibility with water and cannot dissolve the polymer such as alcohol, acetone, or ether.

The fibrous filter substrate or the loose fibers having the hydrophilic polymer without acidic functional groups deposited thereon is then dried. The drying can take place at any suitable temperature and length of time. Suitable temperatures include temperatures of 150° C. or less, or 125° C. or less, or 100° C. or less, or 90° C. or less, or 80° C. or less, or 70° C. or less, or 60° C. or less, or 50° C. or less, or 50° C. to 150° C. The drying temperature should be lower than the melting point of the hydrophilic polymer and the fibrous filter substrate or the loose fibers. Suitable times are from 0.5 h to 24 h, or 0.5 h to 20 h, or 0.5 h to 15 h, or 0.5 h to 12 h, or 0.5 h to 10 h, or 0.5 h to 8 h, or 1 h to 5 h. Typically, the higher the temperature, the lower the time needed.

The hydrophilic polymer without acidic functional groups should be water insoluble, i.e., either the polymer itself is water insoluble or the polymer is rendered insoluble after it interacts with metal ions. For example, chitosan and metal ion-complexed sodium alginate are insoluble in water. In some cases, the hydrophilic polymer without acidic functional groups deposited on the dried fibrous filter substrate or the loose fibers having the hydrophilic polymer without acidic functional groups needs to be converted from a water soluble hydrophilic polymer to a water insoluble hydrophilic polymer by contacting with an aqueous basic or acidic solution by a dip coating or impregnation method. For example, protonated chitosan is water soluble and needs to be converted to water insoluble chitosan. The basic solution may have a concentration in a range of about 0.2 wt % to about 5 wt %, for example. The base used for the preparation of the basic solution can be any base, including, but not limited to, KOH, NaOH, and NH₄OH. The acidic solution may have a concentration in a range of about 0.2 wt % to about 5 wt %, for example. The acid used for the preparation of the acidic solution can be any acid, including, but not limited to, HCl, H₂SO₄, H₃PO₄, and acetic acid.

The virucidal metal, the virucidal metal-containing compound, or combinations thereof is deposited on the surface of the intermingled fibers or the loose fibers having the hydrophilic polymer without acidic functional groups thereon. The amount of the virucidal metal, the virucidal metal-containing compound, or combinations thereof deposited on the surface of the intermingled fibers or the loose fibers having the hydrophilic polymer without acidic functional groups thereon is determined by the Inductively coupled plasma atomic emission spectrometry (ICP-AES) metal analysis method, and is typically in the range of about 0.1 wt % to about 8.0 wt % of the total virucidal fibrous filter, preferably in the range of about 0.1 wt % to about 3.0 wt % of the total virucidal fibrous filter.

The metal or metal-containing compound can be deposited on the surface of the fibers using any suitable method, including, but not limited to, dip coating, impregnating, brushing, spraying, roll coating, spin coating, flow coating, filtration, or combinations thereof. In one embodiment, the virucidal metal, the virucidal metal-containing compound, or combinations thereof is deposited on the surface of the intermingled fibers or the loose fibers having the hydrophilic polymer without acidic functional groups thereon by depositing a metal salt on the surface of the intermingled fibers or the loose fibers having the hydrophilic polymer thereon. The metal salt can be deposited by impregnating the intermingled fibers or the loose fibers having the hydrophilic polymer thereon with a solution of the metal salt. The pH of the metal salt solution is typically less than 7. The metal salt concentration is in the range of about 0.01 wt % to about 5 wt %, or about 0.05 wt % to about 3 wt %. The metal salts can be metal nitrates, metal sulfates, metal phosphates, or metal acetates, for example. The solution of the metal salt may comprise water, alcohol, acetone, ether, or combinations thereof. The treated fibrous filter substrate or the loose fibers are then contacted with an aqueous basic solution having a pH in a range of about 7.5 to about 11 by a dip coating or impregnation method to form a virucial filter media comprising a virucidal metal, a virucidal metal-containing compound, or combinations thereof and the hydrophilic polymer without acidic functional groups deposited thereon. The basic solution has a concentration in a range of about 0.2 wt % to about 5 wt %. The base used for the preparation of the basic solution can be any base, including, but not limited to, KOH, NaOH, or NH₄OH. The fibrous filter substrate or the loose fibers having the virucidal metal, the virucidal metal-containing compound, or combinations thereof and the hydrophilic polymer without acidic functional groups deposited thereon is then dried. The drying can take place at any suitable temperature and length of time, such as those described above.

In another embodiments, the virucidal metal, the virucidal metal-containing compound, or combinations thereof is deposited on the surface of the intermingled fibers or the loose fibers having the hydrophilic polymer without acidic functional groups thereon by depositing a virucidal metal, a virucidal metal-containing compound, or combinations thereof directly on the surface of the intermingled fibers or the loose fibers having the hydrophilic polymer without acidic functional groups thereon. The virucidal metal, the virucidal metal-containing compound, or combinations thereof can be deposited by impregnating the intermingled fibers or the loose fibers having the hydrophilic polymer without acidic functional groups thereon with a suspension of the virucidal metal, the virucidal metal-containing compound, or combinations thereof. The suspension of the virucidal metal, the virucidal metal-containing compound, or combinations thereof can be prepared by adjusting the pH of a metal salt solution from less than 7 to about 7 to about 11 with the addition of a basic solution to the metal salt solution. The metal salt concentration in the metal salt solution is in a range of about 0.01 wt % to about 5 wt %, or about 0.05 wt % to about 3 wt %. The metal salts can be metal nitrates, metal sulfates, metal phosphates, or metal acetates, for example. The solution of the metal salt may comprise water, alcohol, acetone, ether, or combinations thereof. The basic solution may have a concentration in a range of about 0.2 wt % to about 5 wt %. The base used for the preparation of the basic solution can be any base, including, but not limited to, KOH, NaOH, or NH₄OH. The fibrous filter substrate or the loose fibers having the virucidal metal, the virucidal metal-containing compound, or combinations thereof and the hydrophilic polymer without acidic functional groups deposited thereon is then dried. The drying can take place at any suitable temperature and length of time, such as those described above.

In another embodiment, the virucidal metal, the virucidal metal-containing compound, or combinations thereof is deposited on the surface of the intermingled fibers or the loose fibers having the hydrophilic polymer without acidic functional groups thereon by depositing a virucidal metal, a virucidal metal-containing compound, or combinations thereof directly on the surface of the intermingled fibers or the loose fibers having the hydrophilic polymer without acidic functional groups thereon. The virucidal metal, the virucidal metal-containing compound, or combinations thereof can be deposited by impregnating the intermingled fibers or the loose fibers having the hydrophilic polymer without acidic functional groups thereon in a solution of metal salt. While the intermingled fibers or the loose fibers having the hydrophilic polymer without acidic functional groups are in the metal salt solution, the pH is adjusted to maximize the deposition of the virucidal metal, the virucidal metal-containing compound, or combinations thereof onto the intermingled fibers or the loose fibers having the hydrophilic polymer without acidic functional groups. The pH of the mixture of the metal salt solution and the fibers is adjusted to about 7 to about 11 with the addition of a basic solution. The metal salt concentration in the metal salt solution is in a range of about 0.01 wt % to about 5 wt %, or about 0.05 wt % to about 3 wt %. The metal salts can be metal nitrates, metal sulfates, metal phosphates, or metal acetates, for example. The solution of the metal salt may comprise water, alcohol, acetone, ether, or combinations thereof. The basic solution may have a concentration in a range of about 0.2 wt % to about 5 wt %. The base used for the preparation of the basic solution can be any base, including, but not limited to, KOH, NaOH, or NH₄OH. The fibrous filter substrate or the loose fibers having the virucidal metal, the virucidal metal-containing compound, or combinations thereof and the hydrophilic polymer without acidic functional groups deposited thereon is then dried. The drying can take place at any suitable temperature and length of time, such as those described above.

In another embodiment of the method of making the virucidal fibrous filter media, the hydrophilic polymer without acidic functional groups is dissolved in water or an acidic aqueous solution to form an aqueous polymer solution. The acidic aqueous solution can be an acetic acid aqueous solution containing about 0.1 to about 4 wt % of acetic acid in water. In one embodiment, the aqueous polymer solution comprises a polymer with a concentration in a range of about 0.005 to about 5 wt %, or about 0.5 to about 5 wt %. The aqueous polymer solution comprising the hydrophilic polymer without acidic functional groups is mixed with a metal salt solution to form a suspension comprising the hydrophilic polymer and the metal salt having a pH of less than 7, wherein the hydrophilic polymer and the metal salt may form a metal ion/hydrophilic polymer without acidic functional groups complex. The weight ratio of the hydrophilic polymer without acidic functional groups to the metal salt is in a range of 1:15 to 2:1. The pH of the metal salt solution is typically less than 7. The metal salt concentration is in a range of about 0.001 wt % to about 1 wt %, or about 0.01 wt % to about 0.5 wt %. The metal salts can be metal nitrates, metal sulfates, metal phosphates, or metal acetates, for example. The solution of the metal salt may comprise water, alcohol, acetone, ether, or combinations thereof. The suspension comprising the hydrophilic polymer without acidic functional groups and the metal salt is applied onto a fibrous filter substrate comprising a plurality of intermingled fibers or onto loose fibers to deposit the hydrophilic polymer without acidic functional groups and the metal salt onto the surface of the intermingled fibers, or the loose fibers by processes including, but not limited to, dip coating, impregnating, brushing, spraying, roll coating, flow coating, filtration, or combinations thereof. In some cases, prevention of the precipitation of the suspended hydrophilic polymer without acidic functional groups and the metal salt while the suspension is applied onto the fibrous filter substrate may be needed. This can be accomplished by stirring, shaking, ultrasonicating, or combinations thereof, for example. The fibrous filter substrate comprising the hydrophilic polymer without acidic functional groups and the metal salt is contacted with an aqueous basic solution having a pH in a range of about 7.5 to about 11 by a dip coating or impregnation method to form a fibrous filter substrate comprising the hydrophilic polymer without acidic functional group and a virucidal metal, a virucidal metal containing compound, or combinations thereof. The basic solution has a concentration in a range of about 0.2 wt % to about 5 wt %. The base used for the preparation of the basic solution can be any base, including but not limited to, KOH, NaOH, or NH₄OH. The fibrous filter substrate or the loose fibers having the virucidal metal, the virucidal metal-containing compound and the hydrophilic polymer deposited thereon is then dried. The drying can take place at any suitable temperature and length of time, such as those described above.

In yet another embodiment of the method of making the virucidal fibrous filter media, the hydrophilic polymer without acidic functional groups is dissolved in water or an acidic aqueous solution to form an aqueous polymer solution. The acidic aqueous solution can be an acetic acid aqueous solution containing about 0.1 to about 4 wt % of acetic acid in water. In one embodiment, the aqueous polymer solution comprises a polymer with a concentration in a range of about 0.005 to about 5 wt %, or about 0.5 to about 5 wt %. The aqueous polymer solution comprising the hydrophilic polymer without acidic functional groups is mixed with a metal salt solution to form a suspension comprising the hydrophilic polymer without acidic functional groups and the metal salt and a pH of less than 7, wherein the hydrophilic polymer without acidic functional groups and the metal salt may form a metal ion/hydrophilic polymer complex. The weight ratio of the hydrophilic polymer without acidic functional groups to the metal salt is in a range of 1:15 to 2:1. The pH of the metal salt solution is typically less than 7. The metal salt concentration is in a range of about 0.001 wt % to about 1 wt %, or about 0.01 wt % to about 0.5 wt %. The metal salts can be metal nitrates, metal sulfates, metal phosphates, or metal acetates, for example. The solution of the metal salt may comprise water, alcohol, acetone, ether, or combinations thereof. The pH of the suspension was adjusted from less than 7 to about 7 to about 11 with the addition of a basic solution to the suspension to form a suspension comprising the hydrophilic polymer without acidic functional groups and a virucidal metal, a virucidal metal-containing compound, or combinations thereof. The basic solution has a concentration in a range of about 0.2 wt % to about 5 wt %. The base used for the preparation of the basic solution can be any base, including, but not limited to, KOH, NaOH, or NH₄OH.

The suspension comprising the hydrophilic polymer without acidic functional groups, the virucidal metal, the virucidal metal-containing compound, or combinations thereof having a pH in a range of about 7 to about 11 is applied onto a fibrous filter substrate comprising a plurality of intermingled fibers or onto loose fibers to deposit the hydrophilic polymer without acidic functional groups and the virucidal metal, the virucidal metal-containing compound, or combinations thereof onto the surface of the intermingled fibers, or the loose fibers by processes including, but not limited to, dip coating, impregnating, brushing, spraying, roll coating, flow coating, filtration, or combinations thereof. In some cases, prevention of the precipitation of the suspended hydrophilic polymer without acidic functional groups and the virucidal metal, the virucidal metal-containing compound, or combinations thereof while the suspension is applied onto the fibrous filter substrate may be needed. Suitable methods include, but are not limited to, stirring, shaking, ultrasonicating, or combinations thereof, for example.

The fibrous filter substrate or the loose fibers having the virucidal metal, the virucidal metal-containing compound and the hydrophilic polymer without acidic functional groups deposited thereon is then dried. The drying can take place at any suitable temperature and length of time, such as those described above.

An optional step after drying the virucidal filter media prepared using any of the method as mentioned above includes reducing the virucidal filter media using a gas phase reducing agent such as hydrogen or a liquid phase reducing agent such as sodium borohydride (NaBH₄) or hydrazine solution. Reduction treatment of the virucidal metal, the virucidal metal-containing compound, or combinations thereof as an option is to incorporate a long-term release characteristic, wherein virucidal metal cations are slowly created over time via oxidation. In this optional embodiment, either a portion of, or all of, the metal cations are converted to reduced metal to impact long-term release functionality.

It is believed that the present virucidal filter media comprising a fibrous substrate comprising a plurality of intermingled fibers, a hydrophilic polymer without acidic functional groups deposited on a surface of the fibers without forming a continuous coating layer on the substrate, and a virucidal metal, a virucidal metal-containing compound, or combinations thereof deposited on the surface of the fibers comprising the hydrophilic polymer without acidic functional groups has virucidal and/or antibacterial activity and improved virucidal and/or antibacterial activity compared to the filter media deposited with the hydrophilic polymer without acidic functional groups and no virucidal metal, the virucidal metal-containing compound, or combinations thereof. The present virucidal filter media is believed to be beneficial in the fight against many viruses, including the Covid-19 virus. The present virucidal filter media can be used for gas (e.g., air), vapor, or liquid purifications.

By “about,” we mean within 10% of the specified value, or within 5%, or within 1%.

### EXAMPLES

The following examples are provided to illustrate one or more embodiments of the invention, but the invention is not limited to the specific embodiments described. Numerous variations can be made to the following examples that lie within the scope of the invention. The virucidal filter media is expected to have virucidal and/or antibacterial activity or improved virucidal and/or antibacterial activity compared to the filter media deposited with the hydrophilic polymer and no virucidal metal, the virucidal metal-containing compound, or combinations thereof.

### Comparative Example 1: Preparation of Zinc Deposited PP Filter Media

A 0.1 wt % protonated chitosan solution was prepared by dissolving 0.4 g of chitosan in 400 mL of a 1 wt % aqueous acetic acid solution. 3.0 g of Polypropylene (PP) filter media comprising a plurality of intermingled fibers and with a filter performance rating (FPR) rating of 9 was soaked in the 0.1 wt % protonated chitosan solution and shaken for 30 min. The wet fiber media was dried at 80° C. for 1 hour to form a protonated chitosan deposited PP filter media. 0.75 g of zinc nitrate hexahydrate was dissolved in 1 L DI water to form a 0.075 wt % zinc nitrate (Zn(NO₃)₂) aqueous solution. The PP filter media without any treatment was soaked in the 0.075 wt % zinc nitrate (Zn(NO₃)₂) aqueous solution. Then, the pH of the Zn(NO₃)₂ solution was adjusted to 8 using an ammonium hydroxide aqueous solution in the presence of the PP filter media under shaking. The mixture was shaken for 2 hours to deposit virucidal zinc-containing compounds onto the surface of the PP fibers. The zinc treated PP filter media was dried at 80° C. for 1 hour. Finally, the treated filter media was washed with water and dried at 80° C. for 1 hour. Zn ICP-AES analysis results confirmed that the zinc treated PP filter media was deposited with <0.05 wt % of zinc.

### Comparative Example 2: Preparation of Chitosan Treated PP Filter Media (Abbreviated as PP-C)

A 0.1 wt % protonated chitosan solution was prepared by dissolving 0.7 g of chitosan in 700 mL of a 1 wt % aqueous acetic acid solution to form a protonated chitosan solution. PP filter media comprising a plurality of intermingled fibers and with a filter performance rating (FPR) rating of 9 was soaked in the 0.1 wt % protonated chitosan solution and shaken for 30 min. The wet fiber media was dried at 80° C. for 1 hour to form a protonated chitosan deposited PP filter media (PP-C). The PP-C filter media was treated with a 2 wt % NaOH solution to remove the residue acetic acid and convert the protonated water soluble chitosan into water insoluble chitosan.

The virucidal properties of this PP-C filter media against Human Coronavirus strain 229E (ATCC #VR-740) analyzed with ISO 18184:2019(E) show 80.1% reduction after a 4 h exposure against a control propylene filter media sample without any treatment with 0% reduction.

### Example 1: Chitosan/Copper Deposited Polypropylene Filter Media (Abbreviated as PP-C-Cu-A)

A 0.1 wt % protonated chitosan solution was prepared by dissolving 0.7 g of chitosan in 700 mL of a 1 wt % aqueous acetic acid solution to form a protonated chitosan solution. PP filter media comprising a plurality of intermingled fibers and with a filter performance rating (FPR) rating of 9 was soaked in the 0.1 wt % protonated chitosan solution and shaken for 30 min. The wet fiber media was dried at 80° C. for 1 hour to form a protonated chitosan deposited PP filter media (PP-C). The PP-C filter media was treated with a 2 wt % NaOH solution to remove the residue acetic acid and convert the protonated water soluble chitosan into water insoluble chitosan. The treated PP-C filter media was then soaked in an aqueous 0.4 wt % Cu(NO₃)₂ solution and shaken for 30 min to deposit Cu²⁺ onto the surface of the intermingled PP fibers and form chitosan/Cu²⁺ complexes. The treated PP fiber media was further treated with an aqueous 2 wt % NH₄OH solution followed by washing with water and drying at 80° C. for 1 hour to form the final PP-C-Cu-A virucidal filter media. Cu ICPAES analysis results confirmed that PP-C-Cu-A virucidal filter media was deposited with 0.38 wt % of copper.

The virucidal properties of this PP-C-Cu-A virucidal filter media against Human Coronavirus strain 229E (ATCC #VR-740) analyzed with ISO 18184:2019(E) show 99.4% reduction after a 4 h exposure against a control propylene filter media sample without any treatment with 0% reduction.

### Example 2: Chitosan/Copper Deposited Polypropylene Filter Media (Abbreviated as PP-C-Cu-B)

A 0.1 wt % protonated chitosan solution was prepared by dissolving 0.7 g of chitosan in 700 mL of a 1 wt % aqueous acetic acid solution. PP filter media comprising a plurality of intermingled fibers and with a filter performance rating (FPR) rating of 9 was soaked in the 0.1 wt % protonated chitosan solution and shaken for 30 min. The wet fiber media was dried at 80° C. for 1 hour to form a protonated chitosan deposited PP filter media (PP-C). The PP-C filter media was then soaked in an aqueous 0.4 wt % Cu(NO₃)₂ solution for 60 min to deposit Cu′ onto the surface of the intermingled PP fibers and form chitosan/Cu′ complexes. The chitosan and Cu(NO₃)₂ deposited filter media was dried and cured at 80° C. for 1 hour to stabilize the chitosan/Cu′ complexes. The treated PP fiber media was further treated with a 2 wt % NaOH aqueous solution followed by washing with water and drying at 80° C. for 1 hour to form PP-C-Cu-B virucidal filter media. Cu ICP-AES analysis results confirmed that PP-C-Cu-B virucidal filter was deposited with 0.78 wt % of copper.

The virucidal properties of this PP-C-Cu-B virucidal filter media against Human Coronavirus strain 229E (ATCC #VR-740) analyzed with ISO 18184:2019(E) show 99.7% reduction after a 4 h exposure against a control propylene filter media sample without any treatment with 0% reduction.

### Example 3: Chitosan/Zinc Deposited Polypropylene Filter Media (Abbreviated as PP-C-Zn-A)

In this example, a chitosan/zinc complex was formed first and then deposited onto the PP filter media. 150 mg of zinc acetate dihydrate was dissolved in 70 mL of water. 0.21 g of chitosan was dissolved in 6.79 g of 1.0 wt % aqueous acetic acid solution to form a 3 wt % protonated chitosan solution. The protonated chitosan solution was added to the zinc acetate solution slowly under vigorous stirring and stirred for 30 min to form a suspension comprising chitosan/zinc complex. Then 630 mL of ethanol was added to the suspension. The pH of the suspension was adjusted to 8 using a 2 wt % aqueous ammonia. 4.5 g of a PP filter media comprising a plurality of intermingled fibers and with a filter performance rating (FPR) rating of 9 was soaked in the chitosan/zinc complex suspension and sonicated for 1 hour. Thereafter, the treated filter media was dried and cured at 80° C. for 1 hour. Finally, the treated filter media was washed with water to remove any water soluble materials and dried at 80° C. for 1 hour. The final product was designated as PP-C-Zn-A. Zn ICP-AES analysis results confirmed that PP-C-Zn-A virucidal filter was deposited with 0.32 wt % of zinc.

The virucidal properties of this PP-C-Cu-A virucidal filter media against Human Coronavirus strain 229E (ATCC #VR-740) analyzed with ISO 18184:2019(E) show 87.4% reduction after a 4 h exposure against a control propylene filter media sample without any treatment with 0% reduction.

### Example 4: Chitosan/Zinc Deposited Polypropylene Filter Media (Abbreviated as PP-C-Zn-B)

In this example, the chitosan/zinc complex was formed and then deposited on the PP filter media. 260 mg of zinc acetate dihydrate was dissolved in 60 mL water and 540 mL of ethanol to form a zinc acetate solution. 0.18 g of chitosan was dissolved in 5.82 g of 1.0 wt % aqueous acetic acid solution to form a 3 wt % protonated chitosan solution. The zinc acetate solution was added to the protonated chitosan solution slowly under vigorous stirring and stirred for 30 min to form a suspension comprising chitosan/zinc complex. The pH of the mixture was adjusted to 8 using a 2 wt % aqueous ammonia. 4.8 g of a PP filter media comprising a plurality of intermingled fibers and with a filter performance rating (FPR) rating of 9 was soaked in the chitosan/zinc complex suspension and sonicated for 1 hour. Thereafter, the treated filter media was dried and cured at 80° C. for 1 hour. Finally, the treated filter media was washed with water to remove any water soluble materials and dried at 80° C. for 1 hour. The final product was designated as PP-C-Zn-B. Zn ICP-AES analysis results confirmed that PP-C-Zn-B virucidal filter was deposited with 0.42 wt % of zinc.

### Example 5: Chitosan/Zinc Deposited PP Filter Media (Abbreviated as PP-C-Zn-C)

A 0.1 wt % protonated chitosan solution was prepared by dissolving 0.7 g of chitosan in 700 mL of a 1 wt % aqueous acetic acid solution. 5.5 g of PP filter media comprising a plurality of intermingled fibers and with a filter performance rating (FPR) rating of 9 was soaked in the 0.1 wt % protonated chitosan solution and shaken for 30 min. The wet fiber media was dried at 80° C. for 1 hour to form a protonated chitosan deposited PP filter media (PP-C). 4.2 g of zinc nitrate hexahydrate was dissolved in 700 mL DI water to form a 0.6 wt % zinc nitrate aqueous solution. The PP-C filter media was then soaked in the zinc nitrate aqueous solution for 60 min to deposit Zn²⁺ onto the surface of the intermingled PP fibers and form chitosan/Zn²⁺ complexes. The chitosan/zinc deposited filter media was dried and cured at 80° C. for 1 hour. Then the treated PP fiber media was further treated with a NaOH aqueous solution at pH 10 and shaken for 1 hour followed by washing with water and drying at 80° C. for 1 hour. The final product was designated as PP-C-Zn-C. Zn ICP-AES analysis results confirmed that PP-C-Zn-C virucidal filter was deposited with 0.36 wt % of zinc.

### Example 6: Chitosan/Zinc Deposited PP Filter Media (Abbreviated as PP-C-Zn-D)

A 0.1 wt % protonated chitosan solution was prepared by dissolving 0.4 g of chitosan in 400 mL of a 1 wt % aqueous acetic acid solution. 3.0 g of PP filter media comprising a plurality of intermingled fibers and with a filter performance rating (FPR) rating of 9 was soaked in the 0.1 wt % protonated chitosan solution and shaken for 30 min. The wet fiber media was dried at 80° C. for 1 hour to form a protonated chitosan deposited PP filter media. 0.75 g of zinc nitrate hexahydrate was dissolved in 1 L DI water to form a 0.075 wt % zinc nitrate (Zn(NO₃)₂) aqueous solution. The chitosan deposited PP filter media was soaked in the 0.075 wt % zinc nitrate (Zn(NO₃)₂) aqueous solution. Then, the pH of the Zn(NO₃)₂ solution was adjusted to 8 using an ammonium hydroxide aqueous solution in the presence of the chitosan deposited PP filter media under shaking. The mixture was shaken for 2 hours to deposit virucidal zinc-containing compounds onto the surface of the PP fibers. The chitosan/zinc deposited PP filter media was dried and cured at 80° C. for 1 hour. Finally, the treated filter media was washed with water to remove any water-soluble materials and dried at 80° C. for 1 hour. The final product was designated as PP-C-Zn-D. Zn ICP-AES analysis results confirmed that PP-C-Zn-D virucidal filter was deposited with 0.94 wt % of zinc.

The virucidal properties of this PP-C-Zn-D virucidal filter media against SARS-related Coronavirus 2 (SARS-CoV-2) analyzed with ISO 18184:2019(E) show 99.0% reduction after a 4 h exposure against a control propylene filter media sample without any treatment with 0% reduction.

### Specific Embodiments

While the following is described in conjunction with specific embodiments, it will be understood that this description is intended to illustrate and not limit the scope of the preceding description and the appended claims.

A first embodiment of the invention is a virucidal fibrous filter media comprising a fibrous substrate comprising a plurality of intermingled fibers; a hydrophilic polymer without acidic functional groups deposited on a surface of the fibers, wherein the hydrophilic polymer does not form a continuous coating layer on the substrate; and a virucidal metal, a virucidal metal-containing compound, or combinations thereof deposited on the surface of the fibers comprising the hydrophilic polymer. An embodiment of the invention is one, any or all of prior embodiments in this paragraph up through the first embodiment in this paragraph wherein the fibers comprise polyethylene, polypropylene, polyamide, polyacrylonitrile, polyethersulfone, polysulfone, poly(ether ketone), polyimide, polyvinylidene fluoride, carbon, polycarbonate, polyester, cellulose acetate, cellulose triacetate, polybenzimidazole, cellulose, acrylic, glass, or combinations thereof. An embodiment of the invention is one, any or all of prior embodiments in this paragraph up through the first embodiment in this paragraph wherein the hydrophilic polymer comprises a polysaccharide polymer, a polyvinyl alcohol, a poly(ethylene glycol), a poly(vinyl pyrrolidone), a polyacrylamide, a polyethyleneimine, or combinations thereof. An embodiment of the invention is one, any or all of prior embodiments in this paragraph up through the first embodiment in this paragraph wherein the hydrophilic polymer comprises chitosan, sodium carrageenan, potassium carrageenan, sodium alginate, potassium alginate, sodium hyaluronate, potassium hyaluronate, dextran, pullulan, carboxymethyl curdlan, chitin, or combinations thereof. An embodiment of the invention is one, any or all of prior embodiments in this paragraph up through the first embodiment in this paragraph wherein the metal or the metal in the metal-containing compound comprises Ag, Cu, Zn, Ni, Ti, Sn, Bi, W, Fe, Al, Ru, Ir, Mn, Ca, or combinations thereof. An embodiment of the invention is one, any or all of prior embodiments in this paragraph up through the first embodiment in this paragraph wherein the metal-containing compound is a metal oxide, a metal hydroxide, a hydrous metal oxide, a metal sulfate, a hydrated metal sulfate, a metal halide, a hydrated metal halide, a metal nitrate, a hydrated metal nitrate, a hydroxy double salt, or combinations thereof. An embodiment of the invention is one, any or all of prior embodiments in this paragraph up through the first embodiment in this paragraph wherein the hydrophilic polymer comprises chitosan, sodium alginate, potassium alginate, sodium hyaluronate, potassium hyaluronate, polyvinyl alcohol, poly(ethylene glycol), polyethyleneimine, or combinations thereof, and wherein the metal or the metal in the metal-containing compound comprises Ag, Cu, Zn, or combinations thereof.

A second embodiment of the invention is a method of making a virucidal fibrous filter media comprising dissolving a hydrophilic polymer without acidic functional groups in water or an acidic aqueous solution to form an aqueous polymer solution; applying the aqueous polymer solution onto a fibrous filter substrate comprising a plurality of intermingled fibers or onto loose fibers to deposit the hydrophilic polymer on a surface of the intermingled fibers or the loose fibers so that the hydrophilic polymer does not form a continuous coating layer on the substrate; drying the fibrous filter substrate or the loose fibers having the hydrophilic polymer deposited thereon; depositing a virucidal metal, a virucidal metal-containing compound, or combinations thereof on the surface of the intermingled fibers or the loose fibers having the hydrophilic polymer deposited thereon. An embodiment of the invention is one, any or all of prior embodiments in this paragraph up through the second embodiment in this paragraph wherein the intermingled fibers or the loose fibers comprise polyethylene, polypropylene, polyamide, polyacrylonitrile, polyethersulfone, polysulfone, poly(ether ether ketone), polyimide, polyvinylidene fluoride, carbon, polycarbonate, polyester, cellulose acetate, cellulose triacetate, polybenzimidazole, cellulose, acrylic, glass, or combinations thereof. An embodiment of the invention is one, any or all of prior embodiments in this paragraph up through the second embodiment in this paragraph wherein the hydrophilic polymer comprises a polysaccharide polymer, a polyvinyl alcohol, a poly(ethylene glycol), a poly(vinyl pyrrolidone), a polyacrylamide, a polyethyleneimine, or combinations thereof. An embodiment of the invention is one, any or all of prior embodiments in this paragraph up through the second embodiment in this paragraph wherein the hydrophilic polymer comprises chitosan, sodium carrageenan, potassium carrageenan, sodium alginate, potassium alginate, sodium hyaluronate, potassium hyaluronate, dextran, pullulan, carboxymethyl curdlan, chitin, chondroitin, or combinations thereof. An embodiment of the invention is one, any or all of prior embodiments in this paragraph up through the second embodiment in this paragraph wherein the metal or the metal in the metal-containing compound comprises Ag, Cu, Zn, Ni, Ti, Sn, Bi, W, Fe, Al, Ru, Ir, Mn, Ca, or combinations thereof. An embodiment of the invention is one, any or all of prior embodiments in this paragraph up through the second embodiment in this paragraph wherein the metal-containing compound is a metal oxide, a metal hydroxide, a hydrous metal oxide, a metal sulfate, a hydrated metal sulfate, a metal halide, a hydrated metal halide, a metal nitrate, a hydrated metal nitrate, a hydroxy double salt, or combinations thereof. An embodiment of the invention is one, any or all of prior embodiments in this paragraph up through the second embodiment in this paragraph wherein depositing the virucidal metal, the virucidal metal-containing compound, or combinations thereof on the surface of the intermingled fibers or the loose fibers having the hydrophilic polymer thereon comprises depositing a metal salt on the surface of the intermingled fibers or the loose fibers having the hydrophilic polymer thereon; contacting the fibrous filter media deposited with the metal salt and the hydrophilic polymer with a basic solution to form a fibrous filter media having a virucidal metal, a virucidal metal-containing compound, or combinations thereof and the hydrophilic polymer thereon; drying the fibrous filter media having the virucidal metal, the virucidal metal-containing compound, or combinations thereof and the hydrophilic polymer deposited thereon.

A third embodiment of the invention is a method of making a virucidal fibrous filter media comprising dissolving a hydrophilic polymer without acidic functional groups in water or an acidic aqueous solution to form an aqueous polymer solution; mixing the aqueous polymer solution comprising the hydrophilic polymer with a metal salt solution to form a suspension comprising the hydrophilic polymer, and a virucidal metal, a virucidal metal-containing compound, or combinations thereof; applying the suspension onto a fibrous filter substrate comprising a plurality of intermingled fibers or onto loose fibers to deposit the hydrophilic polymer, and the virucidal metal, the virucidal metal-containing compound, or combinations thereof, onto a surface of the intermingled fibers, or the loose fibers; drying the fibrous filter substrate or the loose fibers having the virucidal metal, the virucidal metal-containing compound and the hydrophilic polymer deposited thereon. An embodiment of the invention is one, any or all of prior embodiments in this paragraph up through the third embodiment in this paragraph wherein the fibers comprise polyethylene, polypropylene, polyamide, polyacrylonitrile, polyethersulfone, polysulfone, poly(ether ether ketone), polyimide, polyvinylidene fluoride, carbon, polycarbonate, polyester, cellulose acetate, cellulose triacetate, polybenzimidazole, cellulose, acrylic, glass, or combinations thereof. An embodiment of the invention is one, any or all of prior embodiments in this paragraph up through the third embodiment in this paragraph wherein the hydrophilic polymer comprises a polysaccharide polymer, a polyvinyl alcohol, a poly(ethylene glycol), a poly(vinyl pyrrolidone), a polyacrylamide, a polyethyleneimine, or combinations thereof. An embodiment of the invention is one, any or all of prior embodiments in this paragraph up through the third embodiment in this paragraph wherein the hydrophilic polymer comprises chitosan, sodium carrageenan, potassium carrageenan, sodium alginate, potassium alginate, sodium hyaluronate, potassium hyaluronate, dextran, pullulan, carboxymethyl curdlan, chitin, or combinations thereof. An embodiment of the invention is one, any or all of prior embodiments in this paragraph up through the third embodiment in this paragraph wherein the virucidal metal, the virucidal metal-containing compound, or combinations thereof comprises Ag, Cu, Zn, Ni, Ti, Sn, Bi, W, Fe, Al, Ru, Ir, Mn, Ca, or combinations thereof. An embodiment of the invention is one, any or all of prior embodiments in this paragraph up through the third embodiment in this paragraph wherein a pH of the metal salt solution is in a range of about 6-11.

A fourth embodiment of the invention is a method of making a virucidal fibrous filter media comprising dissolving a hydrophilic polymer without acidic functional groups in water or an acidic aqueous solution to form an aqueous polymer solution; applying the aqueous polymer solution onto a fibrous filter substrate comprising a plurality of intermingled fibers or onto loose fibers to deposit the hydrophilic polymer on a surface of the intermingled fibers or the loose fibers so that the hydrophilic polymer does not form a continuous coating layer on the substrate; drying the fibrous filter substrate or the loose fibers having the hydrophilic polymer deposited thereon; depositing a virucidal metal, a virucidal metal-containing compound, or combinations thereof on the surface of the intermingled fibers or the loose fibers having the hydrophilic polymer deposited thereon. An embodiment of the invention is one, any or all of prior embodiments in this paragraph up through the fourth embodiment in this paragraph wherein the intermingled fibers or the loose fibers comprise polyethylene, polypropylene, polyamide, polyacrylonitrile, polyethersulfone, polysulfone, poly(ether ether ketone), polyimide, polyvinylidene fluoride, carbon, polycarbonate, polyester, cellulose acetate, cellulose triacetate, polybenzimidazole, cellulose, acrylic, glass, or combinations thereof. An embodiment of the invention is one, any or all of prior embodiments in this paragraph up through the fourth embodiment in this paragraph wherein the hydrophilic polymer comprises a polysaccharide polymer, a polyvinyl alcohol, a poly(ethylene glycol), a poly(vinyl pyrrolidone), a polyacrylamide, a polyethyleneimine, or combinations thereof. An embodiment of the invention is one, any or all of prior embodiments in this paragraph up through the fourth embodiment in this paragraph wherein the hydrophilic polymer comprises chitosan, sodium carrageenan, potassium carrageenan, sodium alginate, potassium alginate, sodium hyaluronate, potassium hyaluronate, dextran, pullulan, carboxymethyl curdlan, chitin, chondroitin, or combinations thereof. An embodiment of the invention is one, any or all of prior embodiments in this paragraph up through the fourth embodiment in this paragraph wherein the metal or the metal in the metal-containing compound comprises Ag, Cu, Zn, Ni, Ti, Sn, Bi, W, Fe, Al, Ru, Ir, Mn, Ca, or combinations thereof. An embodiment of the invention is one, any or all of prior embodiments in this paragraph up through the fourth embodiment in this paragraph wherein the metal-containing compound is a metal oxide, a metal hydroxide, a hydrous metal oxide, a metal sulfate, a hydrated metal sulfate, a metal halide, a hydrated metal halide, a metal nitrate, a hydrated metal nitrate, a hydroxy double salt, or combinations thereof. An embodiment of the invention is one, any or all of prior embodiments in this paragraph up through the fourth embodiment in this paragraph wherein depositing the virucidal metal, the virucidal metal-containing compound, or combinations thereof on the surface of the intermingled fibers or the loose fibers having the hydrophilic polymer thereon comprises immersing the surface of the intermingled fibers or the loose fibers having the hydrophilic polymer thereon in a solution of metal salt; adjusting the pH of the mixture of the metal salt solution and the fibers to about 7-11 with the addition of a basic solution to deposit the virucidal metal, the virucidal metal-containing compound, or combinations thereof onto the intermingled fibers or the loose fibers having the hydrophilic polymer without acidic functional groups to form a virucidal fibrous filter media having a virucidal metal, a virucidal metal-containing compound, or combinations thereof and the hydrophilic polymer thereon; drying the fibrous filter media having the virucidal metal, the virucidal metal-containing compound, or combinations thereof and the hydrophilic polymer deposited thereon.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

What is claimed is:

1. A virucidal fibrous filter media comprising:
a fibrous substrate comprising a plurality of intermingled fibers;
a hydrophilic polymer without acidic functional groups deposited on a surface of the fibers, wherein the hydrophilic polymer does not form a continuous coating layer on the substrate; and
a virucidal metal, a virucidal metal-containing compound, or combinations thereof deposited on the surface of the fibers comprising the hydrophilic polymer.

2. The virucidal fibrous filter media of claim 1 wherein the fibers comprise polyethylene, polypropylene, polyamide, polyacrylonitrile, polyethersulfone, polysulfone, poly(ether ether ketone), polyimide, polyvinylidene fluoride, carbon, polycarbonate, polyester, cellulose acetate, cellulose triacetate, polybenzimidazole, cellulose, acrylic, glass, or combinations thereof.

3. The virucidal fibrous filter media of claim 1 wherein the hydrophilic polymer comprises a polysaccharide polymer, a polyvinyl alcohol, a poly(ethylene glycol), a poly(vinyl pyrrolidone), a polyacrylamide, a polyethyleneimine, or combinations thereof.

4. The virucidal fibrous filter media of claim 1 wherein the hydrophilic polymer comprises chitosan, sodium carrageenan, potassium carrageenan, sodium alginate, potassium alginate, sodium hyaluronate, potassium hyaluronate, dextran, pullulan, carboxymethyl curdlan, chitin, or combinations thereof.

5. The virucidal fibrous filter media of claim 1 wherein the metal or the metal in the metal-containing compound comprises Ag, Cu, Zn, Ni, Ti, Sn, Bi, W, Fe, Al, Ru, Ir, Mn, Ca, or combinations thereof.

6. The virucidal fibrous filter media of claim 1 wherein the metal-containing compound is a metal oxide, a metal hydroxide, a hydrous metal oxide, a metal sulfate, a hydrated metal sulfate, a metal halide, a hydrated metal halide, a metal nitrate, a hydrated metal nitrate, a hydroxy double salt, or combinations thereof.

7. The virucidal fibrous filter media of claim 1 wherein the hydrophilic polymer comprises chitosan, sodium alginate, potassium alginate, sodium hyaluronate, potassium hyaluronate, polyvinyl alcohol, poly(ethylene glycol), polyethyleneimine, or combinations thereof, and wherein the metal or the metal in the metal-containing compound comprises Ag, Cu, Zn, or combinations thereof.

8. A method of making a virucidal fibrous filter media comprising:
dissolving a hydrophilic polymer without acidic functional groups in water or an acidic aqueous solution to form an aqueous polymer solution;
applying the aqueous polymer solution onto a fibrous filter substrate comprising a plurality of intermingled fibers or onto loose fibers to deposit the hydrophilic polymer on a surface of the intermingled fibers or the loose fibers so that the hydrophilic polymer does not form a continuous coating layer on the substrate;
drying the fibrous filter substrate or the loose fibers having the hydrophilic polymer deposited thereon;
depositing a virucidal metal, a virucidal metal-containing compound, or combinations thereof on the surface of the intermingled fibers or the loose fibers having the hydrophilic polymer deposited thereon.

9. The method of making a virucidal fibrous filter media of claim 8 wherein the intermingled fibers or the loose fibers comprise polyethylene, polypropylene, polyamide, polyacrylonitrile, polyethersulfone, polysulfone, poly(ether ether ketone), polyimide, polyvinylidene fluoride, carbon, polycarbonate, polyester, cellulose acetate, cellulose triacetate, polybenzimidazole, cellulose, acrylic, glass, or combinations thereof.

10. The method of making a virucidal fibrous filter media of claim 8 wherein the hydrophilic polymer comprises a polysaccharide polymer, a polyvinyl alcohol, a poly(ethylene glycol), a poly(vinyl pyrrolidone), a polyacrylamide, a polyethyleneimine, or combinations thereof.

11. The method of making a virucidal fibrous filter media of claim 8 wherein the hydrophilic polymer comprises chitosan, sodium carrageenan, potassium carrageenan, sodium alginate, potassium alginate, sodium hyaluronate, potassium hyaluronate, dextran, pullulan, carboxymethyl curdlan, chitin, chondroitin, or combinations thereof.

12. The method of making a virucidal fibrous filter media of claim 8 wherein the metal or the metal in the metal-containing compound comprises Ag, Cu, Zn, Ni, Ti, Sn, Bi, W, Fe, Al, Ru, Ir, Mn, Ca, or combinations thereof.

13. The method of making a virucidal fibrous filter media of claim 8 wherein the metal-containing compound is a metal oxide, a metal hydroxide, a hydrous metal oxide, a metal sulfate, a hydrated metal sulfate, a metal halide, a hydrated metal halide, a metal nitrate, a hydrated metal nitrate, a hydroxy double salt, or combinations thereof.

14. The method of making a virucidal fibrous filter media of claim 8 wherein depositing the virucidal metal, the virucidal metal-containing compound, or combinations thereof on the surface of the intermingled fibers or the loose fibers having the hydrophilic polymer thereon comprises:
depositing a metal salt on the surface of the intermingled fibers or the loose fibers having the hydrophilic polymer thereon;
contacting the fibrous filter media deposited with the metal salt and the hydrophilic polymer with a basic solution to form a fibrous filter media having a virucidal metal, a virucidal metal-containing compound, or combinations thereof and the hydrophilic polymer thereon;
drying the fibrous filter media having the virucidal metal, the virucidal metal-containing compound, or combinations thereof and the hydrophilic polymer deposited thereon.

15. The method of making a virucidal fibrous filter media of claim 8 wherein depositing the virucidal metal, the virucidal metal-containing compound, or combinations thereof on the surface of the intermingled fibers or the loose fibers having the hydrophilic polymer thereon comprises:
impregnating the intermingled fibers or the loose fibers having the hydrophilic polymer in a solution of metal salt;
adjusting the pH of the solution of the metal salt in the presence of the intermingled fibers or the loose fibers having the hydrophilic polymer to about 7-11 to deposit the virucidal metal, the virucidal metal-containing compound, or combinations thereof onto the intermingled fibers or the loose fibers having the hydrophilic polymer to form a virucidal fibrous filter media;
drying the fibrous filter media having the virucidal metal, the virucidal metal-containing compound, or combinations thereof and the hydrophilic polymer deposited thereon.

16. A method of making a virucidal fibrous filter media comprising:
dissolving a hydrophilic polymer without acidic functional groups in water or an acidic aqueous solution to form an aqueous polymer solution;
mixing the aqueous polymer solution comprising the hydrophilic polymer with a metal salt solution to form a suspension comprising the hydrophilic polymer, and a virucidal metal, a virucidal metal-containing compound, or combinations thereof;
applying the suspension onto a fibrous filter substrate comprising a plurality of intermingled fibers or onto loose fibers to deposit the hydrophilic polymer, and the virucidal metal, the virucidal metal-containing compound, or combinations thereof, onto a surface of the intermingled fibers, or the loose fibers;
drying the fibrous filter substrate or the loose fibers having the virucidal metal, the virucidal metal-containing compound and the hydrophilic polymer deposited thereon.

17. The method of making a virucidal fibrous filter media of claim 16 wherein the fibers comprise polyethylene, polypropylene, polyamide, polyacrylonitrile, polyethersulfone, polysulfone, poly(ether ether ketone), polyimide, polyvinylidene fluoride, carbon, polycarbonate, polyester, cellulose acetate, cellulose triacetate, polybenzimidazole, cellulose, acrylic, glass, or combinations thereof.

18. The method of making a virucidal fibrous filter media of claim 16 wherein the hydrophilic polymer comprises a polysaccharide polymer, a polyvinyl alcohol, a poly(ethylene glycol), a poly(vinyl pyrrolidone), a polyacrylamide, a polyethyleneimine, or combinations thereof.

19. The method of making a virucidal fibrous filter media of claim 16 wherein the hydrophilic polymer comprises chitosan, sodium carrageenan, potassium carrageenan, sodium alginate, potassium alginate, sodium hyaluronate, potassium hyaluronate, dextran, pullulan, carboxymethyl curdlan, chitin, or combinations thereof.

20. The method of making a virucidal fibrous filter media of claim 16 wherein the virucidal metal, the virucidal metal-containing compound, or combinations thereof comprises Ag, Cu, Zn, Ni, Ti, Sn, Bi, W, Fe, Al, Ru, Ir, Mn, Ca, or combinations thereof.
